(19) United States
(12) Patent Application Publication
(10) Pub. No.: US 2004/0185499 A1
(43) Pub. Date: Sep. 23, 2004

(54) Method of epitope scanning using fluorescence polarization

(76) Inventors: Michael E. Jolley, Round Lake, IL (US); Mohammad Sarwar Nasir, Grayslake, IL (US)

Correspondence Address: Richard A. Machonkin, McDonnell Boehnen Hulbert & Berghoff, 32nd Floor, 300 S. Wacker Drive, Chicago, IL, 60606, US

(21) Appl. No.: 10/393,134

(22) Filed: Mar. 20, 2003

(51) Int. Cl. G01N 33/53

(52) U.S. Cl. 435/7.1

(57) ABSTRACT

An antigenic protein includes a known amino acid sequence. To locate one or more epitopes of the antigenic protein, a plurality of distinct peptides are synthesized bound to respective solid-phase supports via selectively cleavable linkers. Each of the distinct peptides corresponds to a sub-sequence of the antigenic protein's known amino acid sequence. While the peptides are bound to their respective supports, they are conjugated to a fluorophore. The conjugated peptides are then selectively cleaved from their supports, and the fluorescence polarization of the free conjugated peptides is measured. The free conjugated peptides are each combined with an antibody that is able to bind to the antigenic protein, and the fluorescence polarization of the mixtures is measured. A substantial increase in fluorescence polarization of a mixture indicates the presence of an epitope.

### BACKGROUND OF THE INVENTION

1. Field of the Invention

This invention relates to a method for identifying epitopes of antigenic proteins. More particularly, this invention relates to a method of epitope scanning that uses fluorescence polarization.

2. Description of Related Art

An antigenic protein typically has one or more epitopes, which are characterized as regions of the protein that lead to an immune response in an organism. For example, one or more epitopes of an antigenic protein may activate T cells in an organism. This, in turn, may lead to B cell activation, which causes a humoral immune response to be generated (the production of soluble antibodies), and T helper cell activation, which causes a cellular immune response to be generated (the production of a variety of cells which kill the invading organism). The latter pathway is usually activated before the former but is of comparatively limited longevity.

In some cases, an epitope is “sequential” in that it can be defined by a particular sequence of amino acids. In other cases, an epitope is “conformational” in that the epitope is dependent on the three-dimensional structure or conformation of the antigenic protein (e.g., two or more parts of the antigen protein may come together in a particular conformation to form the epitope).

A number of different approaches for identifying the epitopes of an antigenic protein are known. For example, molecular biological approaches, involving cloning, sequencing, restriction enzyme digests and expression, have been used to find epitopes. However, such molecular biological approaches are typically very time consuming and often lack resolution, i.e., the ability to identify which specific amino acids of an antigenic protein correspond to an epitope.

If, however, the amino acid sequence of an antigenic protein is known, then epitope scanning can be used to find the epitopes (or, at least, the sequential epitopes) in the antigenic protein. Certain aspects of epitope scanning are described in Geysen, et al., “Use of peptide synthesis to probe viral antigens for epitopes to a resolution of a single amino acid,” Proc. Nat'l. Acad. Sci. U.S.A., vol. 81, pp. 3998-4002 (1984) and in Geysen, et al., “Strategies for epitope analysis using peptide synthesis,” J. Immunol. Methods, vol. 102, pp. 259-274 (1987), which are incorporated herein by reference.

The epitope scanning process typically involves synthesizing a number of overlapping peptides that correspond to sub-sequences of the antigenic protein's known amino acid sequence. The peptides are usually synthesized attached to a solid-phase support. After synthesis, the peptides are then tested for epitope-related activity using some type of assay, usually ELISA or in vitro lymphocyte activation. Examples of such epitope scanning methods are described in U.S. Pat. Nos. 4,833,092; 5,194,392; 5,539,084; 5,595,915; 5,783,674; and 5,998,577, all of which are incorporated herein by reference.

Conventional epitope scanning methods have a number of disadvantages, however. One problem is that they can be rather labor intensive, usually because of the assays used to screen the peptides. In particular, although techniques exist for synthesizing a large number of different peptides simultaneously, the assays used to screen the peptides can be substantially more involved. For example, although ELISA methods are generally less time consuming than in vitro methods, ELISA methods still typically involve several washings, liquid transfers, and incubation times, making them undesirably labor intensive. Moreover, conventional ELISA methods do not always detect the epitopes that other assay techniques may detect. Thus, the particular assay technique that is used to screen the peptides for epitope scanning may miss epitopes that other assay techniques may find.

Accordingly, there is a need to develop epitope scanning methods that use different assay techniques that may detect epitopes not detected by the assay techniques conventionally used for epitope scanning. In addition, there is a need to develop epitope scanning methods that use assay techniques that can be performed relatively quickly and easily.

### SUMMARY OF THE INVENTION

In a first principal aspect, the present invention provides a method of epitope scanning of an antigenic protein that includes a known amino acid sequence. In accordance with the method, a plurality of distinct amino acid sub-sequences of the known amino acid sequence is identified. A plurality of distinct peptides, each of which corresponds to one of the distinct amino acid sub-sequences, is synthesized. Each of the distinct peptides is conjugated to a fluorophore to provide a plurality of conjugated peptides. Each of the conjugated peptides is combined with an antibody, which antibody is able to bind to the antigenic protein, to provide a plurality of mixtures. The fluorescence polarization of each of the mixtures is measured to obtain a plurality of fluorescence polarization (FP) values.

In a second principal aspect, the present invention provides a method of epitope scanning of an antigenic protein that includes a known amino acid sequence. In accordance with the method, a plurality of distinct amino acid sub-sequences of the known amino acid sequence is identified. A plurality of distinct peptides is synthesized, with each peptide bound to respective solid-phase supports via a selectively cleavable covalent linker. Each of the distinct peptides corresponds to one of the distinct amino acid sub-sequences. A terminal amino group of each of the distinct peptides is conjugated to a fluorophore to provide a plurality of bound conjugated peptides. The bound conjugated peptides are selectively cleaved from their respective solid-phase supports to provide a plurality of free conjugated peptides. The fluorescence polarization of each of the free conjugated peptides is measured to obtain a plurality of initial FP values. The free conjugated peptides are combined with an antibody, which antibody is able to bind to the antigenic protein, to provide a plurality of mixtures. The fluorescence polarization of each of the mixtures is measured to obtain a plurality of final FP values. The final FP values are compared with the initial FP values.

### BRIEF DESCRIPTION OF THE DRAWINGS

FIG. 1 is a graph showing the change in fluorescence polarization measured for various peptides using three different sera containing antibodies to MPB70.

### DETAILED DESCRIPTION OF THE PREFERRED EMBODIMENTS

The preferred embodiments of the present invention provide a method of epitope scanning that uses fluorescence polarization for screening the synthesized peptides. The technique of fluorescence polarization has been successfully utilized in various assays involving proteins, enzymes, drugs, DNA, hormones, peptides and antibodies. The principle behind the fluorescence polarization technique is as follows. Fluorescent probes having a relatively low molecular weight have low fluorescence polarization due to their fast rotation, whereas fluorescent probes with higher molecular weight have a higher fluorescence polarization due to their slower rotation. Thus, the fluorescence polarization of a fluorescent probe often increases upon binding with a target molecule. Further information about the fluorescence polarization technique is provided in Nasir, M. S. and Jolley, M. E., “Fluorescence Polarization: An analytical tool for Immunoassay and Drug Discovery,” Combinatorial Chemistry & High Throughput Screening, vol. 2, pp. 177-190 (1999), which is incorporated herein by reference.

In preferred embodiments of the present invention, the method of epitope scanning may involve the steps of: (1) identifying a plurality of distinct amino acid sub-sequences of the antigenic protein to synthesize as peptides; (2) synthesizing the peptides corresponding to the distinct amino acid sub-sequences; (3) conjugating the peptides with a fluorophore; and (4) screening the conjugated peptides using fluorescence polarization. These steps are described in more detail below.

### 1. Identifying a Plurality of Distinct Amino Acid Sub-sequences of the Antigenic Protein to Synthesize as Peptides

In this step, a known amino acid sequence of an antigenic protein may be consulted to select a number of distinct amino acid sub-sequences to synthesize as peptides in an epitope scanning experiment. In many cases, the known amino acid sequence may be the entire amino acid sequence of the antigenic protein. In other cases, only part of the full amino acid sequence may be known. The sub-sequences that are selected from within the known amino acid sequence may consist of a straight chain of amino acids, or they may be branched. Each amino acid sub-sequence may range in length from two amino acids to nearly the entire known amino acid sequence of the antigenic protein. Typically, the sub-sequences are selected to be at least as long as the epitope of interest is believed to be. On the other hand, when fluorescence polarization measurements are used to screen the peptides, selecting the sub-sequences to be as short as possible can result in higher sensitivity. The amino acid sub-sequences that are selected may all have the same length, or they may have different lengths. In addition, the amino acid sub-sequences may be (but need not be) chosen such that some or all of them are overlapping. For example, the amino acid sub-sequences may be chosen all the same length and offset from each other by a fixed number of amino acids (such as one amino acid, for high resolution) in the known amino acid sequence of the antigenic protein. The amino acid sub-sequences can be chosen to cover the entire known amino acid sequence of the antigenic protein. Alternatively, the amino acid sub-sequences can be chosen to cover only part of the known amino acid sequence, for example, the part believed to contain the epitope of interest.

### 2. Synthesizing the Peptides Corresponding to the Amino Acid Sub-sequences

Once the amino acid sub-sequences are identified, the peptides corresponding to them may be synthesized by any suitable technique. Advantageously, techniques that are able to synthesize a number of different peptides simultaneously may be used. In such high-throughput techniques, the peptides are typically synthesized attached to solid-phase supports, often via a linker. Other techniques for peptide synthesis could be used, however. Typical solid-phase supports include derivatized polyethylene or polypropylene formed into various different shapes, such as “pins” or “gears.” However, other types of solid-phase supports could be used. The linker may be a covalent linker that remains covalently bonded to the solid-phase support and to the peptide while the peptide is being synthesized. The covalent linker may be selectively cleavable to allow the synthesized peptide to be separated from the sold-phase support under relatively mild conditions. Examples of such selectively cleavable linkers are disclosed in U.S. Pat. Nos. 5,539,084 and 5,783,674 and in Maeji, et al., “Multi-pin peptide synthesis strategy for T cell determinant analysis,” J. Immunol. Methods, vol. 134, pp. 23-33 (1990), all of which are incorporated herein by reference. As disclosed therein, the cleavable linker may include a proline residue that cyclizes into a diketopiperazine (DKP) form under mildly basic conditions. This cyclization results in separation of the synthesized peptide from the solid-phase support.

Kits for high-throughput simultaneous peptide synthesis are commercially available. An example is the Multipin™ peptide synthesis kit available from Mimotopes Pty. Ltd. (Clayton, Victoria, Australia). The Multipin™ apparatus includes a reaction tray with 96 wells in which reagents are dispensed, arranged in an 8×12 matrix, and a block that holds 96 “pins” in a corresponding 8×12 matrix. The Multipin™ apparatus may be used with a computer-controlled PinAID™ dispensing aid that uses LEDs to indicate which wells are to receive which reagents in a given cycle. Each “pin” is made up of a “gear,” to which the peptides are coupled during synthesis, and an inert “stem” to which the gear is detachably supported. During synthesis, the block supports the gears so that they are appropriately positioned in the wells.

The gears in such kits are made of polypropylene or polyethylene with the surface derivatized for compatibility with the chemistry used for peptide synthesis. For example, the gears may be radiation grafted with substances to provide functional groups, such as hydroxyl or amine groups, on the surface. The linkers are attached to the functional groups on the gears using appropriate chemistry. Solid-phase supports with linkers already attached are commercially available, such as from Mimotopes Pty. Ltd.

Using these commercially available kits, the peptides may be synthesized in repeated cycles, with one amino acid added in each cycle. In this approach, the terminal amino group in the partially synthesized peptide (or linker, if the first amino acid of the chosen sub-sequence is being added) is protected with a 9-fluorenylmethylcarboxycarbonyl (Fmoc) group at the beginning of each cycle. The solid-phase supports are then Fmoc-deprotected. This can be accomplished by immersing the gears in 20% (v/v) piperidine in dimethylformamide (DMF) followed by washing in DMF and then methanol and then drying. Next, the gears are exposed to the amino acid to be added in the cycle. The amino acid to be added may initially have its α-amino group protected by Fmoc. Certain amino acids may also have side chain protecting groups, such as: t-butyl ether for serine, threonine and tyrosine; t-butyl ester for aspartic acid and glutamic acid; t-butoxycarbonyl for lysine, histidine and tryptophan; 2,2,5,7,8-pentamethylchroman-6-sulfonyl for arginine; and trityl for cysteine. The protected amino acid is activated by adding a solution of 1-hydroxybenzotriazole (HOBT) in DMF, followed by a solution of diisopropylcarbodiimide (DIC) in DMF to provide active amino acid solution. The active amino acid solution is dispensed into the wells to expose the gears. The coupling reaction is allowed to proceed, typically for at least 2 to 4 hours. To complete the cycle, the gears are washed in methanol and then DMF. At the end of the cycle, the Fmoc protecting group of the amino acid that was added becomes the Fmoc-protected terminal amino group of the peptide bound to the gear. Another cycle may then be performed. In this way, successive cycles of amino acid addition may be used to synthesize the desired peptides.

### 3. Conjugating the Peptides With a Fluorophore

After the peptides are completely synthesized, they are conjugated to a fluorophore. If the peptides are synthesized bound to a solid-phase support, as described above, then conjugation may be performed while the peptides are still bound, as described below. To accomplish the fluorophore conjugation, the synthesized peptide is first Fmoc-deprotected as before. The fluorophore is then covalently attached to the terminal amino group using appropriate coupling chemistry. For example, 5-carboxyfluorescein, 6-carboxyfluorescein, or esters thereof, may be attached using DIC/HOBT in DMF.

The fluorophore that is selected for conjugation may depend on the type of linker that is used. For example, DKP-based cleavable linkers cleave spontaneously under mildly basic conditions. However, the covalent attachment of many fluorophores is conducted under basic conditions. Thus, for DKP-based cleavable linkers, fluorophores that can be covalently attached to the terminal amino group under neutral or acidic conditions are preferable. Such fluorophores include 5-carboxyfluorescein, 6-carboxyfluorescein, and esters thereof.

With the fluorophore attached to the terminal amino group, any side chain protecting groups in the peptide may then be removed by using appropriate chemistry. For example, a mixture of trifluoroacetic acid (TFA) and anisole (19:1 v/v) may be used to deprotect many side chain protecting groups.

### 4. Screening the Peptides Using Fluorescence Polarization

The fluorophore-conjugated peptides are then screened using fluorescence polarization to determine which of them, if any, contain the epitope of interest. The fluorescence polarization screening may be performed as follows. If the fluorophore-conjugated peptides are bound to a solid-phase support, they are first separated from the solid-phase support. The use of a selectively cleavable linker greatly facilitates the process, as it allows separation to occur under relatively mild conditions. For example, the DKP-based cleavable linker described above cleaves spontaneously under mildly basic conditions. The cleavage step frees the conjugated peptides, thereby allowing them to be screened using homogeneous assay techniques, such as fluorescence polarization.

The fluorescence polarization of each of the free conjugated peptides is first measured to obtain initial, baseline fluorescence polarization values. Each of the free conjugated peptides is then combined with an appropriate antibody to form a mixture. The mixture is incubated for a period of time and under conditions appropriate to allow binding, if any, to occur. The antibody may be monoclonal or polyclonal. The antibody may be present in natural products, such as blood sera from infected animals. The antibody may be known to bind to a particular epitope of the antigenic protein, or the antibody may be known to bind to the antigenic protein but at an unknown binding site. Alternatively, the binding characteristics of the antibody may be entirely unknown.

After incubation, the fluorescence polarization of each of the mixtures is measured to obtain final polarization values. For each of the peptides, the final fluorescence polarization value is compared to the initial fluorescence polarization value. A substantial increase in fluorescence polarization, i.e., a final polarization value that is substantially greater than the initial fluorescence value, indicates that the peptide contains an epitope to which the antibody binds. In this way, peptide synthesis followed by fluorescence polarization assays to screen the peptides, may be used to locate one or more epitopes of an antigenic protein.

### EXAMPLE

Epitope Scanning of MPB70

MPB70 is an antigenic protein secreted by Mycobacterium bovis. The amino acid sequence of MPB70 is known. For example, the sequence is reported in Radford et al., “Epitope mapping of the Mycobacterium bovis secretory protein MPB70 using overlapping peptide analysis,” J. Gen. Microbiol., vol. 136, pp. 265-272 (1990), which is incorporated herein by reference. Radford, et al. used peptides 8 amino acids in length and overlapping by one amino acid to scan for epitopes in MPB70 using ELISA. Radford, et al. reported finding an epitope to which cattle antibodies responded.

We performed epitope scanning using fluorescence polarization to scan for epitopes in the region of the cattle antibody epitope reported by Radford, et al. Specifically, we identified 96 successive sub-sequences of 15 amino acids, with a spacing of one amino acid, of the following amino acid sequence (which corresponds to amino acids 45 through 154 in the full MPB70 sequence reported by Radford, et al.):

| | |
|---|---|
| 1 NPTGPASVQG MSQDPVAVAA SNNPELTTLT AALSGQLNPQ VNLVDTLNSG QYTVFAPTNA 60 | (SEQ ID NO:1) |
| 61 AFSKLPASTI DELKTNSSLL TSILTYHVVA GQTSPANVVG TRQTLQGASV 110 | |
| (Asn Pro Thr Gly Pro Ala Ser Val Gln Gly Met Ser Gln Asp Pro Val Ala Val Ala | |
| Ala Ser Asn Asn Pro Gln Leu Thr Thr Leu Thr Ala Ala Leu Ser Gly Gln Leu Asn | |
| Pro Gln Val Asn Leu Val Asp Thr Leu Asn Ser Gly Gln Tyr Thr Val Phe Ala Pro | |
| Thr Asn Ala Ala Phe Ser Lys Leu Pro Ala Ser Thr Ile Asp Glu Leu Lys Thr Asn | |
| Ser Ser Leu Leu Thr Ser Ile Leu Thr Tyr His Val Val Ala Gly Gln Thr Ser Pro | |
| Ala Asn Val Val Gly Thr Arg Gln Thr Leu Gln Gly Ala Ser Val) | |

We synthesized the 96 peptides corresponding to the 96 amino acid sub-sequences using a Multipin™ peptide synthesis kit and a PinAID™ dispensing aid. A computer program kept track of weights, volumes and dispensing of various amino acids during the peptide synthesis, and a schedule for the synthesis of peptides using the PinAID™ dispensing aid was generated. The peptides were synthesized on derivatized polypropylene gears. The gears were purchased from Mimotopes Pty. Ltd. (catalog no. KT-96-0-DKP) and had a cleavable diketopiperazine (DKP) linker (1-2 μmole/gear) on them that was used to covalently attach the peptides to the gears during synthesis.

Peptide synthesis was carried out in successive cycles, using protected amino acids, as described above. Thus, in each cycle, the gears were Fmoc-deprotected, the amino acids were activated using the DIC/HOBT chemistry described above, and the activated amino acids were dispensed in the wells in which the gears were positioned. In each cycle, 150 μl of 30 mM activated amino acid solution was dispensed into each well. Coupling was allowed to occur overnight.

After peptide synthesis was complete, the gears were washed and Fmoc deprotected. The unprotected terminal amino groups in the peptides were covalently conjugated to 6-carboxyfluorescein (isomer 2) using standard DIC/HOBT coupling chemistry. The coupling reaction was allowed to occur overnight. The entire block was then washed with DMF and methanol, and the side chains were deprotected using TFA/anisole (19/1). The peptides were then cleaved from the gears using a 40% solution of CH₃CN in phosphate buffered saline (pH 7.4).

Each of the free conjugated peptides was then screened for epitope-related activity using fluorescence polarization. The free conjugated peptides (unpurified) were diluted in phosphate buffered saline (pH 7.5) to a concentration equivalent to 1 nM of fluorophore. After this dilution, the fluorescence polarization of each of the free conjugated peptides was measured to obtain initial, baseline FP values. The baseline FP values of the free conjugated peptides in buffer were found to be between 40 and 45 mP. The free conjugated peptides were then tested with a bovine serum sample (in buffer) that was M. bovis positive, i.e., that contained antibodies that would be expected to react with the epitope identified by Radford, et al. Of the 96 peptides that were synthesized and tested in this way, seven peptides exhibited a substantial increase in fluorescence polarization with this serum sample. These seven peptides were peptides 16, 17, 18, 19, 20, 21, and 22 in the series. The amino acid sequences of these peptides is set out below:

| | | |
|---|---|---|
| Peptide 16: | VAVAASNNPELTTLT (Val Ala Val Ala Ala Ser Asn Asn Pro Glu Leu Thr | (SEQ ID NO:2) |
| | Thr Leu Thr) | |
| Peptide 17: | AVAASNNPELTTLTA (Ala Val Ala Ala Ser Asn Asn Pro Glu Leu Thr Thr | (SEQ ID NO:3) |
| | Leu Thr Ala) | |
| Peptide 18: | VAASNNPELTTLTAA (Val Ala Ala Ser Asn Asn Pro Glu Leu Thr Thr Leu | (SEQ ID NO:4) |
| | Thr Ala Ala) | |
| Peptide 19: | AASNNPELTTLTAAL (Ala Ala Ser Asn Asn Pro Glu Leu Thr Thr Leu Thr | (SEQ ID NO:5) |
| | Ala Ala Leu) | |
| Peptide 20: | ASNNPELTTLTAALS (Ala Ser Asn Asn Pro Glu Leu Thr Thr Leu Thr Ala | (SEQ ID NO:6) |
| | Ala Leu Ser) | |
| Peptide 21: | SNNPELTTLTAALSG (Ser Asn Asn Pro Glu Leu Thr Thr Leu Thr Ala Ala | (SEQ ID NO:7) |
| | Leu Ser Gly) | |
| Peptide 22: | NNPELTTLTAALSGQ (Asn Asn Pro Glu Leu Thr Thr Leu Thr Ala Ala | (SEQ ID NO:8) |
| | Leu Ser Gly Gln) | |

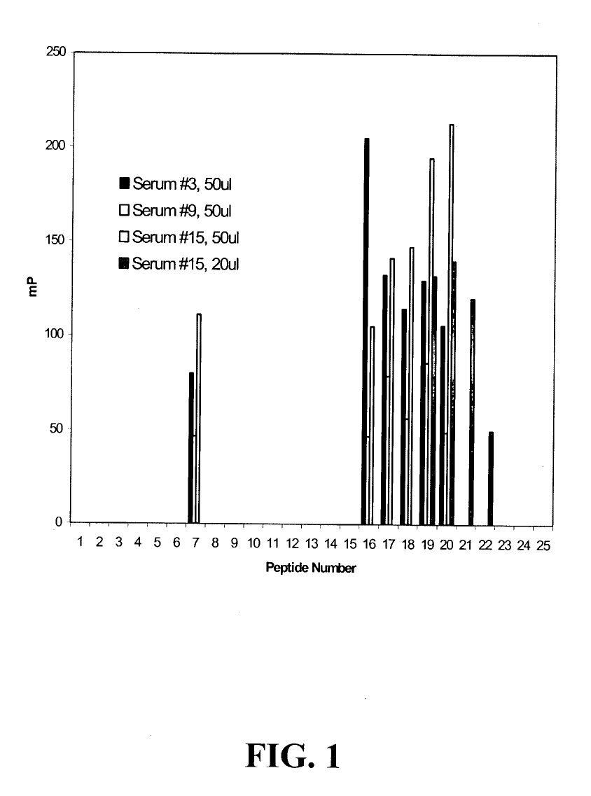

These seven fluorophore-conjugated peptides were purified using HPLC and then tested again. Specifically, the fluorescence polarization values were measured before and after M. bovis positive bovine serum was added. The resulting increases in fluorescence polarization (in mP) for these peptides are plotted in FIG. 1. Three different serum samples were used, identified as serum #3, serum #9, and serum #15. In these tests, a volume of serum (either 50 μl or 20 μl) was added to 1 ml of phosphate buffered saline and combined with free conjugated peptide to a concentration equivalent to 1 nM of fluorophore. Because of a shortage of serum #15, some tests were performed with 20 μl of serum, instead of 50 μl. Each mixture was incubated for a few second at room temperature, and then its fluorescence polarization was measured.

The results shown in FIG. 1 show that peptides 16 through 22 contained one or more epitopes to which bovine serum antibodies are reactive. Although sera from different animals reacted with the peptides differently, these results are generally consistent with the results obtained by Radford, et al. and by others. Significantly, however, these results also show that the fluorescence polarization approach was able to resolve the different reactivities to MPB70 exhibited by antibodies from different animals.

In another experiment, a peptide that was 20 amino acids long was obtained from a commercial source and was tested using these same serum samples in fluorescence polarization assays. The amino acid sequence for this peptide, identified as “peptide 555,” was as follows: SVQGMSQDPVAVAASNNPEL (Ser Val Gln Gly Met Ser Gln Asp Pro Val Ala Val Ala Ala Ser Asn Asn Pro Glu Leu) (SEQ ID:9). The first 15 amino acids of this “peptide 555” correspond to peptide 7 in the series of 96 peptides that were synthesized and screened in the other experiment. In particular, the amino acid sequence for peptide 7 was as follows: SVQGMSQDPVAVAAS (Ser Val Gln Gly Met Ser Gln Asp Pro Val Ala Val Ala Ala Ser) (SEQ ID NO:10). That experiment found that peptide 7 did not result in a significant increase in fluorescence polarization. However, “peptide 555,” with the next 5 amino acids in the sequence, did result in a significant increase in fluorescence polarization when combined with serum samples #3, #9, and #15. The increases in fluorescence polarization (in mP) for “peptide 555” are plotted in FIG. 1 at the peptide number 7 position. These results indicate that the final 5 amino acids in the “peptide 555” sequence contain an epitope to which cattle antibodies are reactive.

These two experiments on MPB70 peptides demonstrate that fluorescence polarization measurements can be used successfully for epitope scanning. Such epitope scanning experiments may involve screening a large number of peptides, as in the first MPB70 experiment, or may involve comparisons between just two peptides, as in the second MPB70 experiment. Other epitope scanning experiments using fluorescence polarization could also be conducted.

The foregoing description of the invention is presented for purposes of illustration and description, and is not intended, nor should be construed, to be exhaustive or to limit the invention to the precise forms disclosed. The description was selected to best explain the principles of the invention and practical application of these principles to enable others skilled in the art to best utilize the invention in various embodiments and with various modifications as are suited to the particular use contemplated. It is intended that the scope of the invention not be limited by the specification, but defined by the claims.

What is claimed is:

1. A method of epitope scanning of an antigenic protein, said antigenic protein including a known amino acid sequence, said method comprising:
identifying a plurality of distinct amino acid sub-sequences of said known amino acid sequence;
synthesizing a plurality of distinct peptides, each of said distinct peptides corresponding to one of said distinct amino acid sub-sequences;
conjugating each of said distinct peptides with a fluorophore to provide a plurality of conjugated peptides;
combining each of said conjugated peptides with an antibody to provide a plurality of mixtures, said antibody being able to bind to said antigenic protein; and
measuring the fluorescence polarization of each of said mixtures to obtain a plurality of mixture fluorescence polarization (FP) values.

2. The method of claim 1, wherein synthesizing a plurality of distinct peptides comprises:
synthesizing each of said distinct peptides bound to a respective solid-phase support.

3. The method of claim 2, wherein synthesizing a plurality of distinct peptides comprises:
synthesizing each of said distinct peptides bound to its respective solid-phase support via a covalent linker.

4. The method of claim 3, wherein said covalent linker is selectively cleavable.

5. The method of claim 4, wherein said covalent linker includes a diketopiperazine-forming moiety.

6. The method of claim 5 wherein said diketopiperazine-forming moiety includes a proline residue.

7. The method of claim 4, wherein conjugating each of said distinct peptides with a fluorophore to provide a plurality of conjugated peptides comprises:
conjugating each of said distinct peptides, while bound to its respective said solid-phase support, with said fluorophore to provide a plurality of bound conjugated peptides.

8. The method of claim 7, further comprising:
selectively cleaving each of said bound conjugated peptides from its respective solid-phase support to provide a plurality of free conjugated peptides.

9. The method of claim 8, wherein combining each of said conjugated peptides with an antibody to provide a plurality of mixtures comprises:
combining each of said free conjugated peptides with said antibody.

10. The method of claim 1, further comprising:
measuring the fluorescence polarization of each of said conjugated peptides to obtain a plurality of conjugate FP values; and
comparing said mixture FP values with said conjugate FP values.

11. The method of claim 1, wherein said distinct amino acid sub-sequences are overlapping.

12. The method of claim 1, wherein said fluorophore is selected from the group consisting of 5-carboxyfluorescein, 6-carboxyfluorescein, and esters thereof.

13. The method of claim 12, wherein said fluorophore is 6-carboxyfluorescein.

14. The method of claim 1, wherein conjugating each of said distinct peptides with a fluorophore to provide a plurality of conjugated peptides comprises:
conjugating a terminal amino group of each of said distinct peptides with a fluorophore to provide a plurality of conjugated peptides.

15. A method of epitope scanning of an antigenic protein, said antigenic protein including a known amino acid sequence, said method comprising:
identifying a plurality of distinct amino acid sub-sequences of said known amino acid sequence;
synthesizing a plurality of distinct peptides bound to respective solid-phase supports via a selectively cleavable covalent linker, each of said distinct peptides corresponding to one of said distinct amino acid sub-sequences;
conjugating a terminal amino group of each of said distinct peptides with a fluorophore to provide a plurality of bound conjugated peptides;
selectively cleaving said bound conjugated peptides from their respective solid-phase supports to provide a plurality of free conjugated peptides;
measuring the fluorescence polarization of each of said free conjugated peptides to obtain a plurality of initial FP values;
combining each of said free conjugated peptides with an antibody to provide a plurality of mixtures, said antibody being able to bind to said antigenic protein;
measuring the fluorescence polarization of each of said mixtures to obtain a plurality of final FP values; and
comparing said final FP values with said initial FP values.

16. The method of claim 15, wherein said selectively cleavable covalent linker includes a diketopiperazine-forming moiety.

17. The method of claim 16, wherein said diketopiperazine-forming moiety includes a proline residue.

18. The method of claim 15, wherein said distinct amino acid sub-sequences are overlapping.

19. The method of claim 15, wherein said fluorophore is selected from the group consisting of 5-carboxyfluorescein, 6-carboxyfluorescein, and esters thereof.

20. The method of claim 19, wherein said fluorophore is 6-carboxyfluorescein.


SEQUENCE LISTING (10 sequences)

SEQ ID NO: 1 - protein, 110 aa, Mycobacterium bovis
NPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASV

SEQ ID NO: 2 - protein, 15 aa, Mycobacterium bovis
VAVAASNNPELTTLT

SEQ ID NO: 3 - protein, 15 aa, Mycobacterium bovis
AVAASNNPELTTLTA

SEQ ID NO: 4 - protein, 15 aa, Mycobacterium bovis
VAASNNPELTTLTAA

SEQ ID NO: 5 - protein, 15 aa, Mycobacterium bovis
AASNNPELTTLTAAL

SEQ ID NO: 6 - protein, 15 aa, Mycobacterium bovis
ASNNPELTTLTAALS

SEQ ID NO: 7 - protein, 15 aa, Mycobacterium bovis
SNNPELTTLTAALSG

SEQ ID NO: 8 - protein, 15 aa, Mycobacterium bovis
NNPELTTLTAALSGQ

SEQ ID NO: 9 - protein, 20 aa, Mycobacterium bovis
SVQGMSQDPVAVAASNNPEL

SEQ ID NO: 10 - protein, 15 aa, Mycobacterium bovis
SVQGMSQDPVAVAAS